(19) United States
(12) Patent Application Publication
(10) Pub. No.: US 2021/0106872 A1
(43) Pub. Date: Apr. 15, 2021

(54) FITNESS MONITORING DEVICE

(71) Applicant: Ayodele Akinremi, Cockeysville, MD (US)

(72) Inventor: Ayodele Akinremi, Cockeysville, MD (US)

(21) Appl. No.: 16/653,511

(22) Filed: Oct. 15, 2019

(51) Int. Cl. A63B 24/00; A61B 5/0205; A61B 5/00

(52) U.S. Cl. CPC A63B 24/0062; A61B 5/0205; A61B 5/681; A61B 5/021; A61B 5/7275; A61B 5/0002; A61B 5/746; A61B 5/7425

(57) ABSTRACT

A fitness monitoring device for optimizing exercise dosage for a user includes a housing. A strap that is coupled to the housing defines a loop that is configured to insert a wrist of a user to couple the housing to the wrist. A first sensor and a second sensor, which are coupled to a lower face of the housing, are configured to contact the wrist of the user. The first sensor and the second sensor are optical type and thus configured to emit light and to measure reflected light to determine a heart rate and blood pressure of the user, respectively, to estimate a fitness state of the user. A screen that is coupled to an upper face of the housing is configured to display to the user the heart rate, the blood pressure, and at least one of an exercise dosage, an exercise recommendation, and the fitness state.

### CROSS-REFERENCE TO RELATED APPLICATIONS

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art including information disclosed under 37 CFR 1.97 and 1.98

The disclosure and prior art relate to monitoring devices and more particularly pertains to a new monitoring device for optimizing exercise dosage for a user.

### BRIEF SUMMARY OF THE INVENTION

An embodiment of the disclosure meets the needs presented above by generally comprising a housing. A strap that is coupled to the housing defines a loop that is configured to insert a wrist of a user to couple the housing to the wrist. A first sensor and a second sensor, which are coupled to a lower face of the housing, are configured to contact the wrist of the user. The first sensor and the second sensor are optical type and thus configured to emit light and to measure reflected light to determine a heart rate and blood pressure of the user, respectively, to estimate a fitness state of the user. A screen that is coupled to an upper face of the housing is configured to display to the user the heart rate, the blood pressure, and at least one of an exercise dosage, an exercise recommendation, and the fitness state.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a fitness monitoring device according to an embodiment of the disclosure.

FIG. 2 is a top view of an embodiment of the disclosure.

FIG. 3 is a bottom view of an embodiment of the disclosure.

FIG. 4 is a side view of an embodiment of the disclosure.

FIG. 5 is a block diagram of an embodiment of the disclosure.

### DETAILED DESCRIPTION OF THE INVENTION

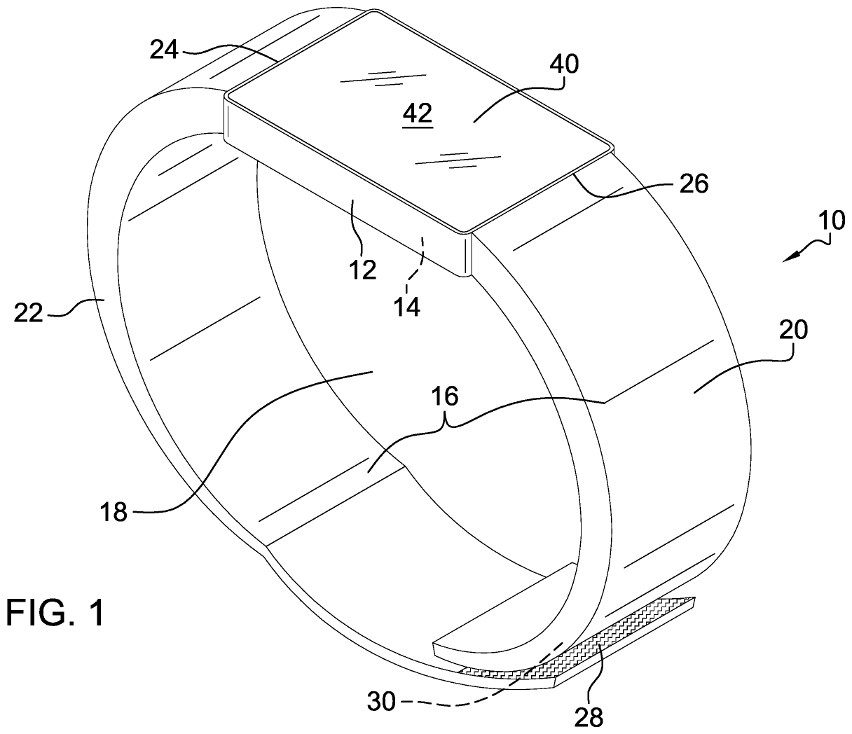

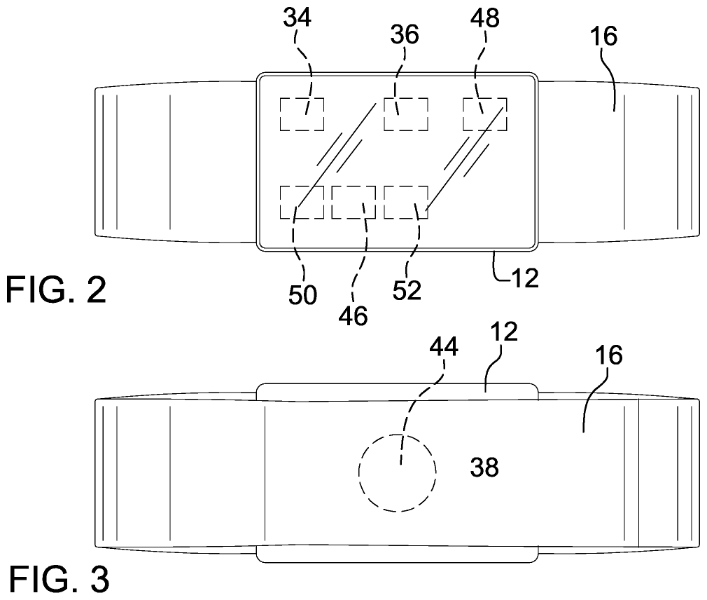

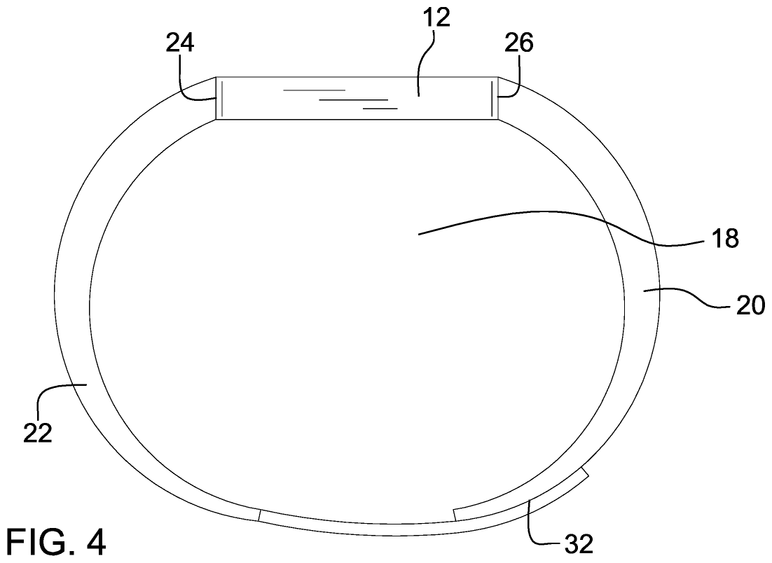

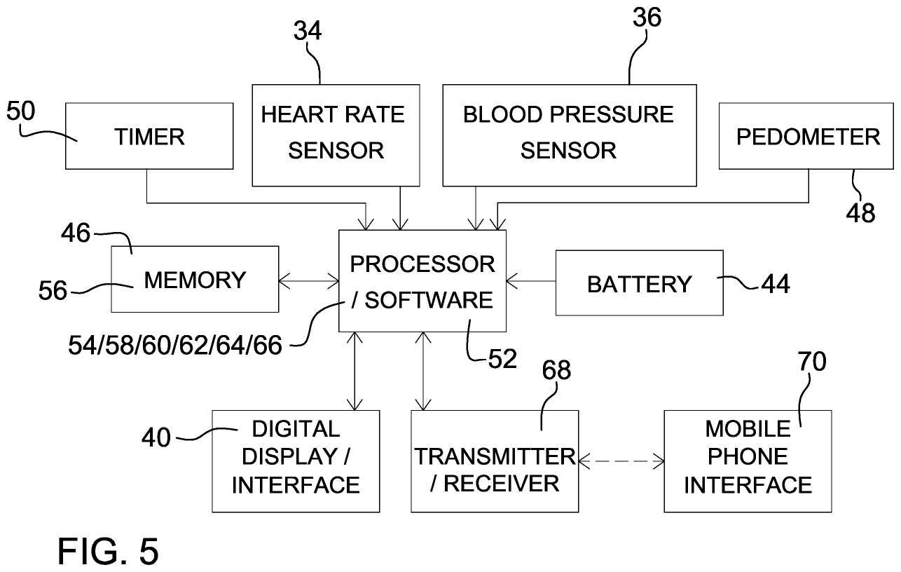

With reference now to the drawings, and in particular to FIGS. 1 through 5 thereof, a new monitoring device embodying the principles and concepts of an embodiment of the disclosure and generally designated by the reference numeral 10 will be described.

As best illustrated in FIGS. 1 through 5, the fitness monitoring device 10 generally comprises a housing 12 that defines an interior space 14. The housing 12 is substantially waterproof. A strap 16 that is coupled to the housing 12 defines a loop 18 that is configured to insert a wrist of a user to couple the housing 12 to the wrist. The strap 16 may comprise a first section 20 and a second section 22, which are coupled to and extend from a first end 24 and a second end 26 of the housing 12, respectively. Alternatively, the housing 12 may be coupled to the wrist by other coupling means, such as, but not limited to, bracelets, adhesives, and the like.

A first connector 28 is coupled to the first section 20 of the strap 16 distal from the housing 12. A second connector 30 is coupled to the second section 22 of the strap 16 distal from the housing 12. The second connector 30 is complementary to the first connector 28 so that the second connector 30 is positioned to selectively couple to the first connector 28 to removably couple the strap 16 around the wrist of the user. The second connector 30 and the first connector 28 may comprise a hook and loop fastener 32, as shown in FIG. 1, or other fastening means, such as, but not limited to, clasps, buckles, and the like.

A first sensor 34 and a second sensor 36, which are coupled to a lower face 38 of the housing 12, are configured to contact the wrist of the user. The first sensor 34 and the second sensor 36 are optical type and thus configured to emit light and to measure reflected light to determine a heart rate and blood pressure of the user, respectively, to estimate a fitness state of the user. The present invention anticipates other sensors being coupled to the housing 12, such as, but not limited to, bioelectric impedance sensors, pulse oximeters, and the like. The present invention also anticipates the first sensor 34 being configured to determine both heart rate and blood pressure, thus eliminating a requirement for the second sensor 36.

A screen 40 that is coupled to an upper face 42 of the housing 12 is configured to display to the user the heart rate, the blood pressure, and at least one of an exercise dosage, an exercise recommendation, and the fitness state. The screen 40 is touch enabled.

A battery 44, a data storage module 46, a pedometer 48, a timer 50, and a microprocessor 52 are coupled to the housing 12 and are positioned in the interior space 14. The microprocessor 52 is operationally coupled to the battery 44, the first sensor 34, the second sensor 36, the screen 40, the data storage module 46, the pedometer 48, and the timer 50.

The pedometer 48 is configured to determine a step count for the user and may be accelerometer type. The timer 50 is configured to determine an elapsed time for a physical activity. The screen 40 is configured to be touched to enter commands into the microprocessor 52 and physiological data into the data storage module 46.

Programming code 54 that is positioned on the microprocessor 52 enables the microprocessor 52 to evaluate inputs from the first sensor 34, the second sensor 36, the pedometer 48, and the timer 50 in conjunction with the physiological data and a plurality of reference tables 56 that are positioned on the data storage module 46 to determine at least one of the exercise parameter and a fitness parameter. The microprocessor 52 is positioned to actuate the screen 40 to display at least one of the exercise dosage, the exercise recommendation, and the fitness state.

The programming code 54 comprises exercise code 58 that enables the microprocessor 52 to calculate the exercise dosage for a selected time period and to actuate the screen 40 to display the exercise dosage for the selected time period. The programming code 54 also comprises alert code 60 that enables the microprocessor 52 to actuate the screen 40 to alert the user when the exercise dosage for the selected time period does not meet a preset value. The exercise code 58 also enables the microprocessor 52 to estimate oxygen consumption based on completion of an exercise, for example a one mile walk as determined by the pedometer 48, within a predetermined time as determined by the timer 50, in conjunction with physiological data of the user.

The programming code 54 also comprises risk code 62 that enables the microprocessor 52 to evaluate respective physiological data relative to associated reference tables 56 to determine at least one of cardiorespiratory fitness, cardiovascular disease risk, and Type II diabetes risk and to selectively actuate the screen 40 to display the cardiorespiratory fitness, the cardiovascular disease risk, and the Type II diabetes risk.

The programming code 54 also comprises BMI code 64 that enables the microprocessor 52 to calculate a baseline body mass index for the user based on associated physiological data and to calculate a change from the baseline body mass index based on changes in the associated physiological data and to actuate the screen 40 to display the baseline body mass index and the change from the baseline body mass index. The BMI code 64 may comprise an algorithm based on the United States Navy Fitness Formula, or other algorithms for estimating percent body fat and basal metabolic rate based on physiological data.

The programming code 54 also comprises evaluation code 66 that enables the microprocessor 52 to evaluate respective physiological data relative to associated reference tables 56 to determine an exercise readiness value, a recommended exercise dosage, and an exercise program for the user and to actuate the screen 40 to display the exercise readiness value, the recommended exercise dosage, and the exercise program. The plurality of reference tables 56 may include Framingham Risk Score tables, as well as tables from National Heart, Lung, and Blood Institute, the American Diabetes Association, Office of Disease Prevention and Health Promotion, and other sources.

A transceiver 68 is coupled to the housing 12 and is positioned in the interior space 14. The transceiver 68 is operationally coupled to the microprocessor 52 so that the microprocessor 52 is positioned to actuate the transceiver 68 to communicate with an electronic device.

Emulation code 70 that is positioned on the electronic device enables the electronic device to emulate the screen 40 on a display of the electronic device. The emulation code 70 allows the user to type the commands and the physiological data on the display so that the electronic device is positioned to communicate the commands and the physiological data to the microprocessor 52, via the transceiver 68, positioning the microprocessor 52 to send the physiological data to the data storage module 46.

In use, the user enters baseline physiological data into the data storage module 46 via the screen 40. The housing 12 then is coupled to the wrist of the user using the hook and loop fastener 32 on the strap 16. The first sensor 34 and the second sensor 36 are configured to determine the heart rate and the blood pressure of the user, respectively, allowing the microprocessor 52 to compare these values, in conjunction with the physiological data and the tables, to estimate a fitness state of the user, and to recommend, via the screen 40, an exercise dosage/routine. The device 10 also apprises the user of cardiorespiratory fitness, cardiovascular disease risk, and Type II diabetes risk.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. A fitness monitoring device comprising:
a housing defining an interior space;
a strap coupled to the housing defining a loop wherein the loop is configured for inserting a wrist of a user for coupling the housing to the wrist;
a first sensor and a second sensor coupled to a lower face of the housing wherein the first sensor and the second sensor are configured for contacting the wrist of the user, the first sensor and the second sensor being optical type wherein the first sensor and the second sensor are configured for emitting light and measuring reflected light for determining a heart rate and blood pressure of the user, respectively, for estimating a fitness state of the user; and
a screen coupled to an upper face of the housing wherein the screen is configured for displaying to the user the heart rate, the blood pressure, and at least one of an exercise dosage, an exercise recommendation, and the fitness state.

2. The device of claim 1, further including the housing being substantially waterproof.

3. The device of claim 1, further comprising:
the strap comprising a first section and a second section, the first section and the second section being coupled to and extending from a first end and a second end of the housing, respectively;
a first connector coupled to the first section of the strap distal from the housing; and
a second connector coupled to the second section of the strap distal from the housing, the second connector being complementary to the first connector such that the second connector is positioned for selectively coupling to the first connector for removably coupling the strap around the wrist of the user.

4. The device of claim 3, further including the second connector and the first connector comprising a hook and loop fastener.

5. The device of claim 1, further comprising:
a battery coupled to the housing and positioned in the interior space;
a data storage module coupled to the housing and positioned in the interior space;
a plurality of reference tables positioned on the data storage module;
a pedometer coupled to the housing and positioned in the interior space wherein the pedometer is configured for determining a step count for and a distance covered by the user;
a timer coupled to the housing and positioned in the interior space wherein the timer is configured for determining an elapsed time for a physical activity;
a microprocessor coupled to the housing and positioned in the interior space, the microprocessor being operationally coupled to the first sensor, the second sensor, the screen, the battery, the data storage module, the pedometer, and the timer wherein the screen is configured for touching for entering commands into the microprocessor and physiological data into the data storage module; and
programming code positioned on the microprocessor enabling the microprocessor for evaluating inputs from the first sensor, the second sensor, the pedometer, and the timer in conjunction with the physiological data and the reference tables positioned on the data storage module for determining at least one of the exercise dosage, the exercise recommendation, and the fitness state positioning the microprocessor for actuating the screen for displaying at least one of the exercise dosage, the exercise recommendation, and the fitness state.

6. The device of claim 5, further including the pedometer being accelerometer type.

7. The device of claim 5, further including the programming code including exercise code enabling the microprocessor for calculating an exercise dosage for a selected time period and for actuating the screen for displaying the exercise dosage for the selected time period.

8. The device of claim 7, further including the programming code including alert code enabling the microprocessor for actuating the screen for alerting the user when the exercise dosage for the selected time period does not meet a preset value.

9. The device of claim 5, further including the programming code including risk code enabling the microprocessor for evaluating respective physiological data relative to associated reference tables for determining at least one of a current and a change from baseline of at least one of cardiorespiratory fitness, cardiovascular disease risk, and Type II diabetes risk and for selectively actuating the screen for displaying at least one of the current and the change from baseline of at least one of the cardiorespiratory fitness, the cardiovascular disease risk, and the Type II diabetes risk.

10. The device of claim 5, further including the programming code including BMI code enabling the microprocessor for calculating a baseline body mass index for the user based on associated physiological data and for calculating a change from the baseline body mass index based on changes in the associated physiological data and for actuating the screen for displaying the baseline body mass index and the change from the baseline body mass index.

11. The device of claim 5, further including the programming code including evaluation code enabling the microprocessor for evaluating respective physiological data relative to associated reference tables for determining an exercise readiness value, a recommended exercise dosage, and an exercise program for the user and for actuating the screen for displaying the exercise readiness value, the recommended exercise dosage, and the exercise program.

12. The device of claim 5, further comprising:
a transceiver coupled to the housing and positioned in the interior space, the transceiver being operationally coupled to the microprocessor such that the microprocessor is positioned for actuating the transceiver for communicating with an electronic device; and
emulation code positioned on the electronic device enabling the electronic device for emulating the screen on a display of the electronic device such that the user is positioned for typing the commands and the physiological data on the display such that the electronic device is positioned for communicating the commands and the physiological data to the microprocessor via the transceiver positioning the microprocessor for sending the physiological data to the data storage module.

13. A fitness monitoring device comprising:
a housing defining an interior space, the housing being substantially waterproof;
a strap coupled to the housing defining a loop wherein the loop is configured for inserting a wrist of a user for coupling the housing to the wrist, the strap comprising a first section and a second section, the first section and the second section being coupled to and extending from a first end and a second end of the housing, respectively;
a first connector coupled to the first section of the strap distal from the housing;
a second connector coupled to the second section of the strap distal from the housing, the second connector being complementary to the first connector such that the second connector is positioned for selectively coupling to the first connector for removably coupling the strap around the wrist of the user, the second connector and the first connector comprising a hook and loop fastener;
a first sensor and a second sensor coupled to a lower face of the housing wherein the first sensor and the second sensor are configured for contacting the wrist of the user, the first sensor and the second sensor being optical type wherein the first sensor and the second sensor are configured for emitting light and measuring reflected light for determining a heart rate and blood pressure of the user, respectively, for estimating a fitness state of the user;
a screen coupled to an upper face of the housing wherein the screen is configured for displaying to the user the heart rate, the blood pressure, and at least one of an exercise dosage, an exercise recommendation, and the fitness state, the screen being touch enabled;
a battery coupled to the housing and positioned in the interior space;
a data storage module coupled to the housing and positioned in the interior space;
a plurality of reference tables positioned on the data storage module;
a pedometer coupled to the housing and positioned in the interior space wherein the pedometer is configured for determining a step count for and a distance covered by the user, the pedometer being accelerometer type;
a timer coupled to the housing and positioned in the interior space wherein the timer is configured for determining an elapsed time for a physical activity;
a microprocessor coupled to the housing and positioned in the interior space, the microprocessor being operationally coupled to the first sensor, the second sensor, the screen, the battery, the data storage module, the pedometer, and the timer wherein the screen is configured for touching for entering commands into the microprocessor and physiological data into the data storage module;
programming code positioned on the microprocessor enabling the microprocessor for evaluating inputs from the first sensor, the second sensor, the pedometer, and the timer in conjunction with the physiological data and the reference tables positioned on the data storage module for determining at least one of the exercise parameter the a fitness parameter positioning the microprocessor for actuating the screen for displaying at least one of the exercise dosage, the exercise recommendation, and the fitness state, the programming code comprising:
exercise code enabling the microprocessor for calculating an exercise dosage for a selected time period and for actuating the screen for displaying the exercise dosage for the selected time period,
alert code enabling the microprocessor for actuating the screen for alerting the user when the exercise dosage for the selected time period does not meet a preset value,
risk code enabling the microprocessor for evaluating respective physiological data relative to associated reference tables for determining at least one of a current and a change from baseline of at least one of cardiorespiratory fitness, cardiovascular disease risk, and Type II diabetes risk and for selectively actuating the screen for displaying at least one of the current and the change from baseline of at least one of the cardiorespiratory fitness, the cardiovascular disease risk, and the Type II diabetes risk,
BMI code enabling the microprocessor for calculating a baseline body mass index for the user based on associated physiological data and for calculating a change from the baseline body mass index based on changes in the associated physiological data and for actuating the screen for displaying the baseline body mass index and the change from the baseline body mass index, and
evaluation code enabling the microprocessor for evaluating respective physiological data relative to associated reference tables for determining an exercise readiness value, a recommended exercise dosage, and an exercise program for the user and for actuating the screen for displaying the exercise readiness value, the recommended exercise dosage, and the exercise program;
a transceiver coupled to the housing and positioned in the interior space, the transceiver being operationally coupled to the microprocessor such that the microprocessor is positioned for actuating the transceiver for communicating with an electronic device; and
emulation code positioned on the electronic device enabling the electronic device for emulating the screen on a display of the electronic device such that the user is positioned for typing the commands and the physiological data on the display such that the electronic device is positioned for communicating the commands and the physiological data to the microprocessor via the transceiver positioning the microprocessor for sending the physiological data to the data storage module.
